(19) United States
(12) Patent Application Publication
(10) Pub. No.: US 2026/0174312 A1
(43) Pub. Date: Jun. 25, 2026

(54) HORIZONTAL IMAGE ALIGNMENT IN ROTATABLE IMAGING SYSTEM

(71) Applicant: INTUITIVE SURGICAL OPERATIONS, INC., Sunnyvale, CA (US)

(72) Inventors: Peter Forst, Emmendingen (DE); Etienne Holbein, Gundelfingen (DE); Ralf Kleiser, Freiburg (DE); Peter Liebetraut, Gundelfingen (DE); Pink McDowall, San Francisco, CA (US); Max J. Trejo, San Jose, CA (US); Felipe Walker, Freiburg (DE); Manuel Weiner, Emmendingen (DE)

(21) Appl. No.: 19/128,452

(22) PCT Filed: Nov. 9, 2023
(86) PCT No.: PCT/US2023/079175

§ 371 Date: May 8, 2025

Related U.S. Application Data
(60) Provisional application No. 63/382,967, filed on Nov. 9, 2022.

(51) Int. Cl. A61B 1/00; A61B 1/06

(52) U.S. Cl. CPC A61B 1/00006; A61B 1/00009; A61B 1/0004; A61B 1/00045; A61B 1/00066; A61B 1/00179; A61B 1/00183; A61B 1/0655

(57) ABSTRACT

A surgical system includes a handheld cable housing with a user input device positioned thereon. The surgical system includes an endoscope shaft with a distal end and a proximal end. The proximal end of the endoscope shaft is coupled to the handheld cable housing, where the distal end of the endoscope shaft comprises an image sensor. The endoscope shaft is rotatable relative to the handheld cable housing. One or more angular position sensors configured to measure an angular offset relative to a defined image horizon. Upon receiving angular offset data indicative of an angular offset relative to the defined image horizon, an image processor generates rotated image data based on the angular offset data and causes display of the rotated image data.

### CROSS-REFERENCE TO RELATED APPLICATIONS

This application claims priority to and the benefit of U.S. Provisional Patent App. No. 63/382,967, filed Nov. 9, 2022, which is incorporated herein by reference in its entirety.

### BACKGROUND

Conventional endoscopes include an image sensor in a handle of the endoscope and a rotatable rod lens system that relays a scene from a field of view of the rotatable rod lens system to the image sensor. As the rod lens system is rotated to view different fields of view, the rod lens optics rotate relative to the fixed image sensor. Therefore, the image produced by the image sensor remains oriented relative to the fixed image sensor within the endoscope handle.

### SUMMARY

A first aspect of the disclosure includes a surgical system comprising a handheld cable housing with a user input device positioned thereon. The surgical system further comprising an endoscope shaft with a distal end and a proximal end. The proximal end of the endoscope shaft is coupled to the handheld cable housing. The distal end of the endoscope shaft comprises an image sensor. The endoscope shaft is rotatable relative to the handheld cable housing. The surgical system further comprising one or more angular position sensors configured to measure an angular offset relative to a defined image horizon.

In some implementation of the first aspect of the surgical system, the one or more angular position sensors are positioned at a coupling between the endoscope shaft and the handheld cable housing.

In any of the above implementations of the first aspect of the surgical system, the defined image horizon is relative to the handheld cable housing.

In any of the above implementations of the first aspect of the surgical system, the angular offset is a measurement of an angular rotation of the endoscope shaft relative to the handheld cable housing.

In any of the above implementations of the first aspect of the surgical system, the angular offset is a measurement of angular rotation between the defined image horizon and a direction of view of the endoscope shaft.

In any of the above implementations of the first aspect of the surgical system, the defined image horizon is a horizontal midline plane of the handheld cable housing at the coupling.

In any of the above implementations of the first aspect of the surgical system, the defined image horizon is orthogonal to a vertical midline plane of the handheld cable housing and parallel to a longitudinal axis of the handheld cable housing.

In any of the above implementations of the first aspect of the surgical system, the defined image horizon is relative to the endoscope shaft.

In any of the above implementations of the first aspect of the surgical system, the angular offset is a measurement of an angular rotation of the handheld cable housing relative to the endoscope shaft.

In any of the above implementations of the first aspect of the surgical system, the defined image horizon is a horizontal midline plane of the endoscope shaft at the coupling.

In any of the above implementations of the first aspect of the surgical system, the defined image horizon is orthogonal to a vertical midline plane of the endoscope shaft and parallel to a longitudinal axis of the endoscope shaft.

In any of the above implementations of the first aspect of the surgical system, the defined image horizon is based on a sensed direction of gravity.

In any of the above implementations of the first aspect of the surgical system, the one or more angular position sensors are positioned at the distal end of the endoscope shaft.

In any of the above implementations of the first aspect of the surgical system, the one or more angular position sensors are positioned at the handheld cable housing.

In any of the above implementations of the first aspect of the surgical system, the one or more angular position sensors include one or more sensors selected from the group consisting of a hall effect sensor, a mechanical encoder, an optical encoder, a magnetic encoder, an electromagnetic induction encoder, an encoder, a rotary potentiometer, a resolver, a gravity sensor, a gyroscope, a magnetometer, and a linear acceleration sensor.

In any of the above implementations of the first aspect of the surgical system, the user input device is positioned on a control surface of the handheld cable housing.

In any of the above implementations of the first aspect of the surgical system, the control surface is a top surface of the handheld cable housing.

In any of the above implementations of the first aspect of the surgical system, the user input device is selected from a group of user input devices consisting of a physical button, a capacitive sense button, a soft button on a touch screen, a switch, a touch pad, a scroll wheel, and a directional pad.

In any of the above implementations of the first aspect of the surgical system, the handheld cable housing comprises a socket sized and configured to receive the proximal end of the endoscope shaft.

In any of the above implementations of the first aspect of the surgical system, the handheld cable housing comprises a lock configured to maintain the proximal end of the endoscope shaft within the socket of the handheld cable housing.

In any of the above implementations of the first aspect of the surgical system, the lock is biased in a locked configuration.

In any of the above implementations of the first aspect of the surgical system, the handheld cable housing comprises a release lever selectable to configure the lock in an unlocked configuration for releasing the proximal end of the endoscope shaft within the socket of the handheld cable housing.

In any of the above implementations of the first aspect of the surgical system, the handheld cable housing comprises a data interface configured to receive image data from the image sensor and the angular offset from the one or more angular position sensors.

In any of the above implementations of the first aspect of the surgical system, the proximal end of the endoscope shaft comprises a corresponding data interface configured to supply image data from the image sensor and the angular offset from the one or more angular position sensors to the handheld cable housing.

In any of the above implementations of the first aspect of the surgical system, the handheld cable housing comprises a connector cable configured to communicate the image data and the angular offset data to an external device.

In any of the above implementations of the first aspect of the surgical system, the proximal end of the endoscope shaft comprises a housing configured to remain fixed with respect to the handheld cable housing. The proximal end of the endoscope shaft further comprises a rotatable interface configured to facilitate rotation of the endoscope shaft with respect to the handheld cable housing.

In any of the above implementations of the first aspect of the surgical system, the distal end of the endoscope shaft comprises an optical assembly positioned to receive light incident on a distal face of the endoscope shaft.

In any of the above implementations of the first aspect of the surgical system, the distal face is oriented at an angle to the endoscope shaft.

In any of the above implementations of the first aspect of the surgical system, the angle is any angle from 0°-90°.

In any of the above implementations of the first aspect of the surgical system, the optical assembly comprises one or more lenses that direct light incident on the distal face along an optical path to the image sensor.

In any of the above implementations of the first aspect of the surgical system, a direction of view of the endoscope shaft is configured to change in response to rotation of the endoscope shaft relative to the handheld cable housing.

A second aspect of the disclosure includes a method, the method includes defining an image horizon relative to an orientation of a surgical system. The surgical system comprises a handheld cable housing with a user input device positioned thereon and a shaft with an image sensor positioned in a distal end of the shaft. The shaft is coupled to the handheld cable housing such that the shaft is rotatable relative to the handheld cable housing. The method includes determining an angular offset between the image horizon and a direction of view of the shaft. The method includes transmitting image data captured by the image sensor and angular offset data indicative of the angular offset, the angular offset data for rotation of the image data.

In some implementations of the second aspect of the disclosure, the angular offset is measured by one or more angular position sensors of the surgical system.

In any of the above implementations of the second aspect of the method, the one or more angular position sensors are positioned at a coupling between the shaft and the handheld cable housing.

In any of the above implementations of the second aspect of the method, the image horizon is defined as a horizontal midline plane of the handheld cable housing at the coupling.

In any of the above implementations of the second aspect of the method, the image horizon is defined relative to the handheld cable housing.

In any of the above implementations of the second aspect of the method, the angular offset is a measurement of an angular rotation of the shaft relative to the handheld cable housing.

In any of the above implementations of the second aspect of the method, the angular offset is a measurement of angular rotation between the image horizon and a direction of view of the image sensor.

In any of the above implementations of the second aspect of the method, the image horizon is defined as orthogonal to a vertical midline plane of the handheld cable housing and parallel to a longitudinal axis of the handheld cable housing.

In any of the above implementations of the second aspect of the method, the image horizon is defined relative to the shaft.

In any of the above implementations of the second aspect of the method, the angular offset is a measurement of an angular rotation of the handheld cable housing relative to the shaft.

In any of the above implementations of the second aspect of the method, the image horizon is defined as a horizontal midline plane of the shaft at the coupling.

In any of the above implementations of the second aspect of the method, the image horizon is defined as orthogonal to a vertical midline plane of the shaft and parallel to a longitudinal axis of the shaft.

In any of the above implementations of the second aspect of the method, the image horizon is defined based on a sensed direction of gravity.

In any of the above implementations of the second aspect of the method, the one or more angular position sensors are positioned at the shaft.

In any of the above implementations of the second aspect of the method, the one or more angular position sensors are positioned at the handheld cable housing.

In any of the above implementations of the second aspect of the method, the one or more angular position sensors include one or more sensors selected from the group consisting of a hall effect sensor, a mechanical encoder, an optical encoder, a magnetic encoder, an electromagnetic induction encoder, an encoder, a rotary potentiometer, a resolver, a gravity sensor, a gyroscope, a magnetometer, and a linear acceleration sensor.

In any of the above implementations of the second aspect of the method, the user input device is positioned on a control surface of the handheld cable housing.

In any of the above implementations of the second aspect of the method, the control surface is a top surface of the handheld cable housing.

In any of the above implementations of the second aspect of the method, the user input device is selected from a group of user input devices consisting of a physical button, a capacitive sense button, a soft button on a touch screen, a switch, a touch pad, a scroll wheel, and a directional pad.

In any of the above implementations of the second aspect of the method, the handheld cable housing comprises a socket, the socket sized and configured to receive a proximal end of the shaft.

In any of the above implementations of the second aspect of the method, the handheld cable housing comprises a lock configured to maintain the proximal end of the shaft within the socket of the handheld cable housing.

In any of the above implementations of the second aspect of the method, the lock is biased in a locked configuration.

In any of the above implementations of the second aspect of the method, the handheld cable housing comprises a release lever selectable to configure the lock in an unlocked configuration for releasing the proximal end of the shaft within the socket of the handheld cable housing.

In any of the above implementations of the second aspect of the method, the handheld cable housing comprises a data interface configured to receive the image data and the angular offset data.

In any of the above implementations of the second aspect of the method, the proximal end of the shaft comprises a corresponding data interface. The method includes transmitting the image data and the angular offset data from the shaft to the handheld cable housing.

In any of the above implementations of the second aspect of the method, the handheld cable housing comprises a connector cable. The method includes transmitting the image data and the angular offset data from the handheld cable housing to an external device.

A third aspect of the disclosure includes a method, the method includes receiving image data captured by an image sensor positioned in a distal end of a shaft. The shaft is coupled to a handheld cable housing. A user input device is positioned on the handheld cable housing. The shaft is rotatable relative to the handheld cable housing. The method includes receiving angular offset data indicative of an angular offset relative to a defined image horizon. The method includes generating rotated image data based on the angular offset data. The method includes causing display of the rotated image data.

In some implementations of the third aspect of the disclosure, the method includes receiving a control signal in response to selection of the user input device. The control signal sets the defined image horizon to one of a plurality of image horizons.

In any of the above implementations of the third aspect of the disclosure, the method includes supplying light from an illumination source to the handheld cable housing.

In any of the above implementations of the third aspect of the disclosure, the method includes receiving a second control signal in response to selection of the user input device. The second control signal causes the light from the illumination source to change.

In any of the above implementations of the third aspect of the disclosure, the second control signal causes the light from the illumination source to turn off or change frequency.

In any of the above implementations of the third aspect of the disclosure, causing display of the rotated image data comprises transmitting the rotated image data to an external display.

In any of the above implementations of the third aspect of the disclosure, the defined image horizon is relative to the handheld cable housing.

In any of the above implementations of the third aspect of the disclosure, the angular offset is a measurement of an angular rotation of the shaft relative to the handheld cable housing.

In any of the above implementations of the third aspect of the disclosure, the angular offset is a measurement of angular rotation between the defined image horizon and a direction of view of the image sensor.

In any of the above implementations of the third aspect of the disclosure, the defined image horizon is a horizontal midline plane of the handheld cable housing at a coupling between the shaft and the handheld cable housing.

In any of the above implementations of the third aspect of the disclosure, the defined image horizon is orthogonal to a vertical midline plane of the handheld cable housing and parallel to a longitudinal axis of the handheld cable housing.

In any of the above implementations of the third aspect of the disclosure, the defined image horizon is relative to the shaft.

In any of the above implementations of the third aspect of the disclosure, the angular offset is a measurement of an angular rotation of the handheld cable housing relative to the shaft.

In any of the above implementations of the third aspect of the disclosure, the defined image horizon is a horizontal midline plane of the shaft at a coupling between the shaft and the handheld cable housing.

In any of the above implementations of the third aspect of the disclosure, the defined image horizon is orthogonal to a vertical midline plane of the shaft and parallel to a longitudinal axis of the shaft.

In any of the above implementations of the third aspect of the disclosure, the defined image horizon is based on a sensed direction of gravity.

In any of the above implementations of the third aspect of the disclosure, one or more angular position sensors for measuring the angular offset are positioned at the shaft.

In any of the above implementations of the third aspect of the disclosure, one or more angular position sensors for measuring the angular offset are positioned at the handheld cable housing.

In any of the above implementations of the third aspect of the disclosure, one or more angular position sensors configured to measure the angular offset are selected from the group consisting of a hall effect sensor, a mechanical encoder, an optical encoder, a magnetic encoder, an electromagnetic induction encoder, an encoder, a rotary potentiometer, a resolver, a gravity sensor, a gyroscope, a magnetometer, and a linear acceleration sensor.

In any of the above implementations of the third aspect of the disclosure, the user input device is positioned on a control surface of the handheld cable housing.

In any of the above implementations of the third aspect of the disclosure, the control surface is a top surface of the handheld cable housing.

In any of the above implementations of the third aspect of the disclosure, the user input device is selected from a group of user input devices consisting of a physical button, a capacitive sense button, a soft button on a touch screen, a switch, a touch pad, a scroll wheel, and a directional pad.

These and other features will be more clearly understood from the following detailed description taken in conjunction with the accompanying drawings and claims.

### BRIEF DESCRIPTION OF THE DRAWINGS

For a more complete understanding of the present disclosure, reference is now made to the following brief description, taken in connection with the accompanying drawings and detailed description, wherein like reference numerals represent like parts.

FIG. 1 is a plan view of a minimally invasive teleoperated surgical system.

FIG. 2 is a perspective view of a user control system.

FIG. 3 is a perspective view of an electronics cart.

FIG. 4 is a diagrammatic illustration of a teleoperated surgical system.

FIG. 5 is a cross-sectional view of a chip-in-tip (CIT) endoscopic image capture device.

FIGS. 6A-6B show an endoscopic image capture device with a rotatable endoscope assembly positioned to capture images from different fields of view.

FIG. 7 is a block diagram of elements of an endoscope with a rotatable endoscope assembly coupled to a fixed handheld cable housing.

FIGS. 8A-8B show the endoscopic image capture device with different options for defining an image horizon.

FIG. 9 is a diagram of image processing operations to maintain horizontal image alignment.

FIG. 10A is a cross-sectional view of an example endoscope showing a coupling between a fixed handheld cable housing and a rotatable endoscope assembly.

FIG. 10B is an exploded view of the rotatable endoscope assembly of FIG. 10A.

FIG. 10C is a perspective view of the rotatable endoscope assembly of FIG. 10A showing details of an angular position sensor for measuring rotation between the rotatable endoscope assembly and the fixed handheld cable housing.

FIG. 11 is a flowchart of operation of an image capture device according to various implementations described herein.

FIG. 12 is a flowchart of operation of an image processor according to various implementations described herein.

FIG. 13 illustrates an exemplary computer system.

### DETAILED DESCRIPTION

It should be understood at the outset that although illustrative implementations of one or more embodiments are illustrated below, the disclosed systems and methods may be implemented using any number of techniques, whether currently known or in existence. The disclosure should in no way be limited to the illustrative implementations, drawings, and techniques illustrated below, but may be modified within the scope of the appended claims along with their full scope of equivalents. Use of the phrase “and/or” indicates that any one or any combination of a list of options can be used. For example, “A, B, and/or C” means “A”, or “B”, or “C”, or “A and B”, or “A and C”, or “B and C”, or “A and B and C”.

Elements described in detail with reference to one embodiment, implementation, or application may, whenever practical, be included in other embodiments, implementations, or applications in which they are not specifically shown or described. For example, if an element is described in detail with reference to one embodiment and is not described with reference to a second embodiment, the element may nevertheless be claimed as included in the second embodiment. Thus, to avoid unnecessary repetition in the following description, one or more elements shown and described in association with one embodiment, implementation, or application may be incorporated into other embodiments, implementations, or aspects unless specifically described otherwise, unless the one or more elements would make an embodiment or implementation non-functional, or unless two or more of the elements provide conflicting functions.

Some implementations are described in terms of an implementation using a da Vinci™ surgical system (such as as the da Vinci™ Xi™ surgical system), commercialized by Intuitive Surgical, Inc. of Sunnyvale, California. Knowledgeable persons will understand, however, that inventive aspects disclosed herein may be embodied and implemented in various ways, including robotic and, if applicable, non-robotic embodiments and implementations. Implementations on da Vinci™ surgical systems are merely exemplary and are not to be considered as limiting the scope of the inventive aspects disclosed herein.

In accordance with various aspects, the present disclosure describes a system for maintaining an image horizon in a chip-in-tip (CIT) endoscopic image capture device with a rotatable endoscope assembly with rigid or flexible shaft (e.g., a rotatable endoscope shaft). A distal end of the shaft of the rotatable endoscope assembly comprises a camera tip with imaging optics and one or more image sensors, collectively or singularly referred to as an image sensor. The rotatable endoscope assembly is coupled (releasably or fixedly) to a handheld cable housing and is rotatable relative thereto.

The rotatable endoscope assembly is moved by an alignment wheel or lever attached to the shaft. For example, the rotatable endoscope assembly may be rotatable at angles of +/−180° relative to a mid-point position, rotatable +360° relative to a start position, rotatable −360° relative to an end position, or any other intermediate position between the start and the end position or any subset of angles. In some implementations, the rotatable endoscope assembly does not include a start or end position such that the rotatable endoscope assembly can rotate in either direction without limit (e.g., continuously rotatable).

One or more angular position sensors, collectively or singularly referred to as an angular position sensor, measures an angular offset relative to a defined horizon as the rotatable endoscope assembly is rotated relative to the handheld cable housing. The horizon may be defined relative to the orientation of the handheld cable housing, relative to gravity as sensed in the distal tip of the rotatable endoscope assembly (e.g., at the camera tip), gravity as sensed by a fixed proximal end of the rotatable endoscope assembly that connects with the handheld cable housing, gravity as sensed by the handheld cable housing, and/or relative to a user-defined horizon.

The handheld cable housing comprises one or more control buttons, collectively or singularly referred to as a control button. The control button is a physical button, a capacitive sense button, a soft button on a touch screen, switch, touch pad, scroll wheel, directional pad, or any other user input device. The control button is positioned on a control surface of the handheld cable housing such that the control button is easily accessible even when the rotatable endoscope assembly is rotated relative to the handheld cable housing. This is in contrast to endoscopic image capture devices where one or more control buttons may be positioned at a proximal end of the rotatable endoscope assembly. In such systems, the control buttons rotate with the rotatable endoscope assembly so that access to the control button changes as the rotatable endoscope assembly rotates, requiring additional dexterity to activate the control buttons as their position changes over time.

Therefore, according to the present disclosure, access to the control button remains the same even as a direction-of-view (DOV) of the rotatable endoscope assembly changes upon rotation. For example, when the control surface is positioned on a top surface of the handheld cable housing, the handheld cable housing may be maintained in a vertical orientation for optimum visibility of the control surface and accessibility to the control button in support of single-hand control without a need for twisting the wrist, leading to an ergonomic benefit. In other implementations, the control surface is positioned on one or more other surfaces of the handheld cable housing, such as a side surface, a grip of the handheld cable housing, a surface that extends from a top of a grip of the handheld cable housing, or any other surface on or extending from the handheld cable housing.

The rotatable endoscope assembly receives power and illumination from the handheld cable housing. The rotatable endoscope assembly comprises a fiber optic bundle with one or more optical fibers configured to convey light received from the handheld cable housing to the camera tip to illuminate a scene being imaged by the image sensor, such as a diagnostic or surgical procedure. Alternatively, light may illuminate the scene as provided by a single optical fiber, a phosphorus conversion layer at the camera tip, multiple single optical fibers that transport individual or combine wavelengths of light, or one or more illumination sources, such as light emitting diodes (e.g., white or color multiplex), positioned at the camera tip. The rotatable endoscope shaft supplies still or video images captured by the image sensor and one or more signals indicative of the angular offset measured by the angular position sensor to the handheld cable housing.

The handheld cable housing comprises a flexible cable with a second fiber optic bundle with one or more optical fibers. The cable comprises a connector configured to couple the second fiber optic bundle to a light source. In some implementations, the light source is positioned external to the handheld cable housing. In some implementations, the light source is positioned in the handheld cable housing. Images captured by the image sensor in the camera tip are conveyed via a wired or wireless electrical or optical connection to the handheld cable housing and in turn conveyed via a wired or wireless electrical or optical connection in the flexible cable to the connector.

The control surface with the control button is positioned between the flexible cable and the rotatable endoscope assembly. Upon selection of the control button, a control signal is conveyed via a wired or wireless electrical or optical connection in the flexible cable to the connector. The control signal provides instructions to turn on or off an illumination source, capture a still image from the image sensor, start/stop video recording, define an image horizon, turn horizontal image alignment on or off, or perform any other control function for operation of the endoscopic image capture device.

A controller system, such as an electronics cart, comprises a socket configured to accept the connector. The controller system comprises a light source coupled to the socket and configured to supply light to the second fiber optic bundle in the flexible cable. Alternatively, the controller system comprises a power supply for generating illumination in the handheld cable housing or at the camera tip, such as via one or more light emitting diodes. The controller system also comprises an image processor coupled to socket and configured to receive the images and angular offset measurements conveyed via the electrical or optical connection in the flexible cable. In some implementations, the angular offset is encoded as metadata within the video feed or still image. In some implementations, the angular offset is communicated as a separate file that may include a timestamp or reference to a video frame or still image to which the angular offset is associated.

The controller system is coupled to a local and/or remote monitor and configured to display the images processed by the image processor. The controller system is further configured to receive and process the control signal, such as by the image processor or another processor. For example, the controller system operates to turn on/off the illumination source, store a still image from the image sensor, store video data, and/or store the defined horizon.

The image processor is configured to rotate the received images based on the received angular offset measurements to maintain a constant image horizon in images displayed on the monitor (e.g., perform horizontal image alignment). Therefore, even as the image provided by the image sensor rotates with changes in the DOV of the rotatable endoscope assembly, the image processor aligns the images to the defined image horizon before being displayed on a monitor.

While the various examples provided herein are described with respect to an endoscopic image capture device, the pending disclosure is not so limited and is intended to encompass any device coupled to the controller system configured to rotate the received images based on the received angular offset measurements to maintain a constant image horizon in images displayed on the monitor. Likewise, the pending disclosure is intended to encompass any image capture device that is coupled to a controller system for processing images captured by the image capture device. For example, the pending disclosure may equally apply to a borescope or other such inspection camera.

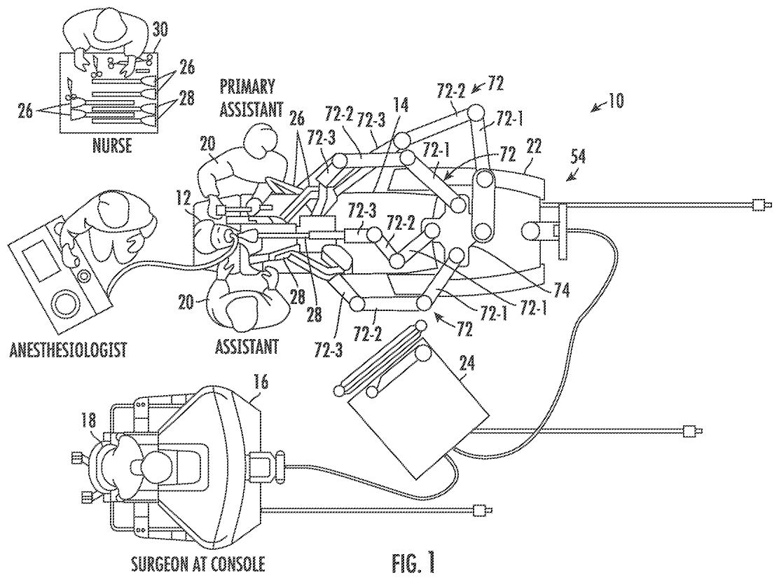

Referring now to the drawings, in which like reference numerals represent like parts throughout the several views, FIG. 1 is a plan view of a minimally invasive teleoperated surgical system 10, typically used for performing a minimally invasive diagnostic or surgical procedure on a patient 12 who is lying on a mobile operating table 14. The system includes a user control system 16, such as a mobile surgeon's console for use by a surgeon 18 during the procedure. One or more assistants 20 may also participate in the procedure. The minimally invasive teleoperated surgical system 10 further includes a manipulating system 22, such as a mobile patient-side cart, and a mobile electronics cart 24. In some embodiments, the mobile operating table 14, user control system 16, manipulating system 22, and the electronics cart 24 are wheel mounted to provide mobility.

The manipulating system 22 or other such manipulating system includes multiple segmented mechanical support arms 72, each having one end portion rotatably mounted to a vertical support structure 74 and having another end mounting a removably coupled surgical instrument 26. In some of embodiments, each mechanical support arm 72 includes a first segment 72-1, a second segment 72-2 and a third segment 72-3. During setup for a procedure, the multiple segments of at least one support arm 72 are moved to position a surgical instrument for insertion within a minimally invasive incision in the body of the patient 12.

During the procedure, while instruments are inserted within a patient's body cavity, the surgeon 18 views the surgical site through the user control system 16. An image of the surgical site can be obtained by an endoscope 28, such as a stereoscopic endoscope, which can be manipulated by the manipulating system 22 to orient the endoscope 28. In some implementations the manipulating system 22 may manipulate the rotatable endoscope assembly (e.g., rotatable endoscope shaft) to change a direction of view of the endoscope 28.

In some implementations, the endoscope 28 may be implemented as the endoscopic image capture device described above with the rotatable endoscope assembly with a distal end comprising a CIT image sensor and a proximal end that is coupled (releasably or fixedly) to a handheld cable housing such that the rotatable endoscope assembly is rotatable relative to the handheld cable housing. The endoscope 28 captures video or still image data and uses one or more angular position sensors to measure an angular offset relative to a defined horizon as the rotatable endoscope assembly is rotated relative to the handheld cable housing.

In some implementations, the surgeon 18 manually manipulates the endoscope 28 within a patient's body cavity. The surgeon 18 views the surgical site through a monitor, such as a monitor on the electronics cart 24 or another monitor external to the electronics cart 24. In such implementations, the surgeon 18 manually manipulates the rotatable endoscope assembly (e.g., via an alignment wheel or lever attached thereto) to change a direction of view of the endoscope 28 while maintaining ergonomic access to the control button on the handheld cable housing. The surgeon 18 may also manipulate control functions on the endoscope 28 via manipulation of the control button to change one or more operating functions (e.g., turn on or off illumination, change illumination source, etc.) of the electronics cart 24 or image processing functions (e.g., rotation of received images to an image horizon, capture still image, etc.) performed by the electronics cart 24.

Computer processor(s) located on the electronics cart 24 can be used to process the images of the surgical site for subsequent display to the surgeon 18 through the user control system 16 or another display, such as on the electronics cart 24. The computer processor(s) may alternatively be referred to herein as an image processor or video processor. M ore generally, the image processor or video processor referenced throughout the disclosure refer to any processor capable of performing the image or video processing functionality described herein, inclusive of a general purpose processor, graphics processor, video processor, image processor, or application specific integrated circuit, for example. The image processor is configured to rotate received images based on the angular offset measurement received from the endoscope 28 to maintain a constant image horizon in images displayed on the user control system 16 or another display.

One or more illumination sources or illuminators may also be provided on the electronics cart 24 to provide light for use by the endoscope 28 for illuminating the surgical site. The illuminators may include a white light source, a colored light source (e.g., red, green, blue, cyan, magenta, yellow, etc.), an infrared light source, a laser light source, or any other type of light source or combination thereof. Different illuminators may be used at different points in time in a surgical or diagnostic procedure. For example, the electronics cart 24 may be controlled, such as through a selection on the user control system 16 or the endoscope 28 (e.g., via the control button), to provide light from a first set of one or more of the illuminators at a first time and provide light from a second set of one or more of the illuminators at a second time.

The number of surgical instruments 26 used at one time will generally depend on the diagnostic or surgical procedure and the space constraints within the operating room among other factors. If it is necessary to change one or more of the surgical instruments 26 being used during a procedure, an assistant 20 can remove the surgical instrument 26 from the manipulating system 22, and replace it with another surgical instrument 26 from a tray 30 in the operating room.

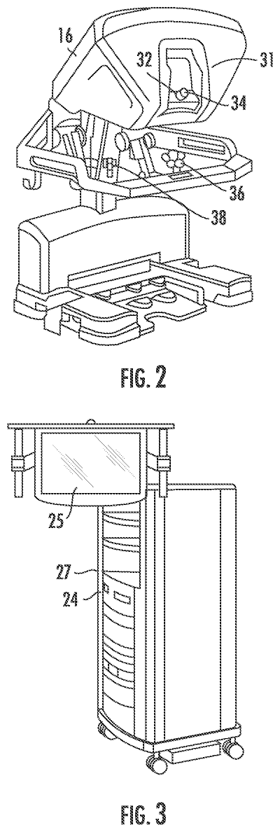

FIG. 2 is a perspective view of the user control system 16. The user control system 16 includes a display area 31 with a left eye display 32 and a right eye display 34 for presenting the surgeon 18 with a coordinated stereoscopic view of the surgical site that enables depth perception.

The user control system 16 further includes one or more control inputs 36. One or more surgical instruments installed for use on the manipulating system 22 (shown in FIG. 1) move in response to surgeon 18′s manipulation of the one or more control inputs 36. The control inputs 36 can provide the same mechanical degrees of freedom as their associated surgical instruments 26 (shown in FIG. 1) to provide the surgeon 18 with telepresence, or the perception that the control inputs 36 are integral with the instruments 26 so that the surgeon has a strong sense of directly controlling the instruments 26. To this end, position, force, and tactile feedback sensors (not shown) may be employed to transmit position, force, and tactile sensations from the surgical instruments 26 back to the surgeon's hands through the control inputs 36. A height of the control inputs 36 may be adjusted with a height adjustment lever 38.

The user control system 16 is usually located in the same room as the patient so that the surgeon can directly monitor the procedure, be physically present if necessary, and speak to a patient-side assistant directly rather than over the telephone or other communication medium. But, the surgeon can be located in a different room, a completely different building, or other remote location from the patient allowing for remote surgical procedures.

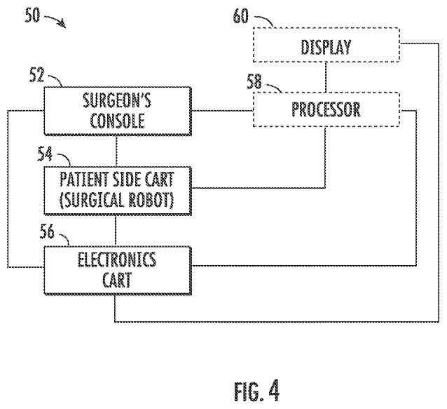

FIG. 3 is a perspective view of the electronics cart 24. The electronics cart 24 can be coupled with the endoscope 28 and includes a computer processor to process captured images for subsequent display, such as to a surgeon on the user control system 16, or on another suitable display located locally and/or remotely. For example, if a stereoscopic endoscope is used, a computer processor on electronics cart 24 can process the captured images to present the surgeon with coordinated stereo images of the surgical site. Such coordination can include alignment between the opposing images and can include adjusting the stereo working distance of the stereoscopic endoscope.

As another example, image processing can include to rotate received images based on received angular offset measurements to maintain a constant image horizon in images displayed on a display 25 of the electronics cart 24 or displayed by the display area 31 of the user control system 16.

Optionally, equipment in electronics cart 24 may be integrated into the user control system 16 or the manipulating system 22, or it may be distributed in various other locations in the operating room. More generally, the electronics cart 24 or user control system 16 with the integrated equipment from the electronics cart 24 may be referred to herein as a controller system for receiving angular offset measurements and rotating images from the image capture device to maintain a constant displayed image horizon.

FIG. 4 diagrammatically illustrates a teleoperated surgical system 50 (such as the minimally invasive teleoperated surgical system 10 of FIG. 1). A user control system 52 (such as user control system 16 in FIG. 1) can be used by a surgeon to control a manipulating system 54 (such as manipulating system 22 in FIG. 1) during a minimally invasive procedure. The manipulating system 54 can use an image capture device, such as a stereoscopic endoscope, to capture images of a surgical site and output the captured images to a computer processor located on an electronics cart 56 (such as the electronics cart 24 in FIG. 1).

The computer processor on the electronics cart 56 typically includes one or more data processing boards purposed for executing computer readable code stored in a non-volatile memory device of the computer processor. As with the electronics cart 24, the electronics cart 56 also includes one or more illumination sources for supplying light to the image capture device.

In some implementations, the user control system 52 and manipulating system 54 are omitted and a surgeon manually manipulates an endoscope (such as endoscope 28 in FIG. 1). The endoscope 28 captures images of a surgical site and output the captured images to a computer processor located on an electronics cart 56.

In one aspect, the computer processor can process the captured images in a variety of ways prior to any subsequent display. For example, the computer processor can use angular offset measurements to rotate images from the image capture device to maintain a constant displayed image horizon. Additionally or in the alternative, the captured images can undergo image processing by a computer processor located outside of electronics cart 56.

In one aspect, teleoperated surgical system 50 includes an optional computer processor 58 (as indicated by dashed line) similar to the computer processor located on electronics cart 56, and manipulating system 54 outputs the captured images to computer processor 58 for image processing prior to display on the user control system 52. In another aspect, captured images first undergo image processing by the computer processor on electronics cart 56 and then undergo additional image processing by computer processor 58 prior to display on the user control system 52 or a display 60.

Teleoperated surgical system 50 can include an optional display 60, as indicated by dashed line. Display 60 is coupled with the computer processor located on the electronics cart 56 and with computer processor 58, and captured images processed by these computer processors can be displayed on display 60 in addition to being displayed on a display of the user control system 52. In various implementations, the display 60 may be located on the electronics cart 56, such as with the display 25 on the electronics cart 24. In some implementations, the display 60 may be separate from the user control system 52 and the electronics cart 58.

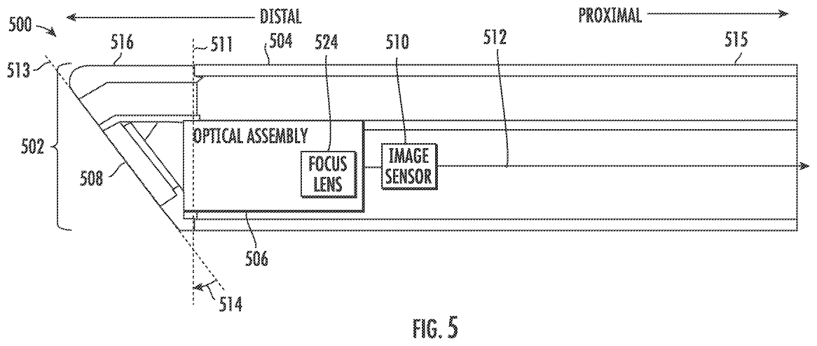

FIG. 5 is a cross-sectional view of a chip-in-tip (CIT) image capture device 500 according to various implementations. The CIT image capture device 500 is positioned on a distal end 502 of a shaft 504 of the CIT image capture device 500. In some implementations, the CIT image capture device 500 is used in the endoscope 28, described above. The CIT image capture device 500 comprises an optical assembly 506 positioned to receive light incident on a distal face 508 of the CIT image capture device 500.

In the example shown, the distal face 508 is at an angle 514 formed between a plane 511 that is orthogonal to the shaft 504 and a plane 513 that is parallel to the distal face 508 (e.g., 10°, 20°, 30°, 45°, or any other desired angle from 0°-90°). In some implementations, the distal face 508 is orthogonal to the shaft 504 (i.e., the angle 514 is 0°). In some implementations, the distal face 508 is parallel to the shaft 504 (i.e., the angle 514 is 90°).

The optical assembly 506 comprises one or more lenses that direct the incident light along an optical path to an image sensor 510. The image sensor 510 captures still and/or video images of a scene (e.g., a surgical site) and communicates the captured images along a wired or wireless communication pathway 512.

In the example shown in FIG. 5, the image sensor 510 is shown as a single image sensor. In some implementations, more than one image sensor may be positioned within the optical pathway. In some implementations, the CIT image capture device 500 comprises a plurality of optical pathways, each of which direct light to one or more image sensors.

For example, the CIT image capture device 500 may include a stereoscopic capture device with a left and right optical pathway and one or more image sensors positioned along each of the left and right optical pathways. In some implementations, a different number of image sensors may be used on each of the left and right optical pathways.

In the example shown in FIG. 5, the communication pathway 512 is a wired communication pathway within the shaft 504 of the CIT image capture device 500. For example, the wired communication pathway 512 may be with a wire, wire bundle, cable, shielded cable, flat flex, or any other wired communication pathway.

In some implementations, the CIT image capture device 500 includes an illumination element 515, such as a light pipe, single optical fiber, or fiber optic bundle. The illumination element 515 directs light from an illumination source to illumination optics 516 in the distal end 502 of the shaft 504 of the CIT image capture device 500. The illumination optics 516 direct the light from the illumination source to illuminate the scene being captured by the image sensor 510. While a single illumination element 515 is shown in the example of FIG. 5, it is contemplated that multiple illumination elements may be present for supplying light from a plurality of illumination sources. In some implementations, light may illuminate the scene being captured by the image sensor 510 as provided by a single optical fiber, a phosphorus conversion layer at the camera tip, multiple single optical fibers that transport individual or combine wavelengths of light, or one or more illumination sources, such as light emitting diodes (e.g., white or color multiplex), positioned at the camera tip.

Each of the features of the CIT image capture device 500 described above may be used separately or in combination with one another or other features described throughout this disclosure. Various modifications and additions to the CIT image capture device 500 are readily discernable by those of ordinary skill in the art and are contemplated by this disclosure. For example, alternative illumination, filtering, optical assembly, focus manipulation, and image sensor features known to those of ordinary skill in the art are contemplated by this disclosure.

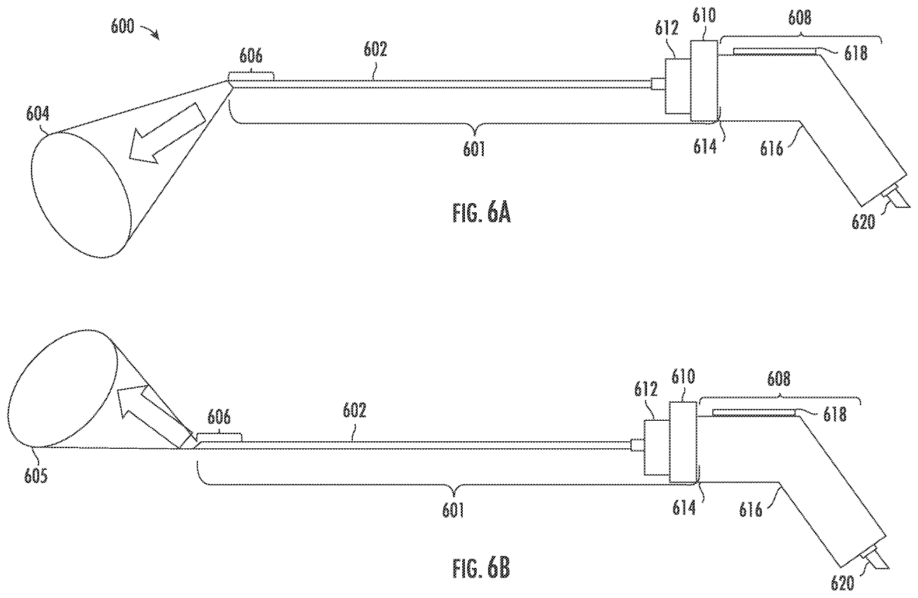

FIGS. 6A-6B show an endoscopic image capture device 600 with a rotatable endoscope assembly 601 (e.g., rotatable endoscope shaft) positioned to capture images from different fields of view (FOV). In various implementations, the endoscopic image capture device 600 is used as the endoscope 28, described above.

As shown in FIG. 6A, shows the endoscopic image capture device 600 with the rotatable endoscope assembly 601 positioned to capture images from a first FOV. FIG. 6B, shows the endoscopic image capture device 600 with the rotatable endoscope assembly 601 positioned to capture images from a second FOV 605. In the example shown, the second FOV 605 is 180° from the first FOV 604, though the different FOVs may be at any angle.

The rotatable endoscope assembly 601 comprises a CIT image capture device 606, such as the CIT image capture device 500, described above. The rotatable endoscope assembly 601 comprises a shaft 602, such as the shaft 504 described above. The shaft 602 is a rigid shaft, flexible shaft, partially flexible shaft, steerable flexible shaft, steerable rigid shaft, or any combination thereof. In some implementations, the CIT image capture device 606 is positioned on a steerable tip of the rotatable endoscope shaft 602.

The rotatable endoscope assembly 601 is coupled to a handheld cable housing 608 and is rotatable relative thereto. The rotatable endoscope assembly 601 comprises a lever 610 to facilitate rotation of the rotatable endoscope assembly 601 relative to the handheld cable housing 608.

In the example shown, the lever 610 is an alignment wheel with a plurality of ergonomic protrusions to facilitate rotation via a finder or thumb of a user. In some implementations, the lever 610 may simply be a single arm that extends from the rotatable endoscope assembly 601 for providing leverage to rotate the rotatable endoscope assembly 601. Other variations of the lever 610 are contemplated by this disclosure.

The rotatable endoscope assembly 601 is rotatable relative to a reference position (e.g., a home position). For example, the rotatable endoscope assembly 601 may be rotatable at angles of +/−180° relative to a mid-point position, rotatable +360° relative to a start position, rotatable −360° relative to an end position, or have the reference position at any other intermediate position between the start and the end position and be rotatable across any subset of angles. In some implementations, the rotatable endoscope assembly 601 does not include a stop such that the rotatable endoscope assembly can rotate in either direction without limit (e.g., continuously rotatable).

In some implementations, the lever 610 includes a physical feature indicative of the reference position. In the example shown in FIGS. 6A & 6B, the protrusions of the lever 610 form a pentagonal shape whereby the central protrusion of the pentagonal shape is indicative of the reference position. Other physical features indicative of the reference position, such as a notch, line, colored stripe, or the like on the lever 610 or the rotatable endoscope shaft 602, are contemplated by this disclosure.

In some implementations, the handheld cable housing 608 also includes a physical feature indicative of the reference position. For example, the handheld cable housing 608 may include a notch, line, or colored stripe that corresponds to the physical feature on the lever 610.

In some implementations, the rotatable endoscope assembly 601 comprises a focus wheel 612 to facilitate manipulation of a focus of the endoscopic image capture device 600. For example, with reference to FIG. 5, the focus wheel 612 may manipulate a focus lens 524 within the optical path of the optical assembly 506 to change the focus of the CIT image capture device 500 in response to manipulation of the focus wheel 612.

The handheld cable housing 608 comprises a coupling 614 that connects the handheld cable housing 608 with the rotatable endoscope assembly 601. The coupling 614 facilitates wired and/or wired transmission of data and power between the rotatable endoscope assembly 601 and the handheld cable housing 608. For example, the coupling 614 may supply from the handheld cable housing 608 to power the CIT image capture device 606 in the rotatable endoscope assembly 601. Likewise, the coupling 614 facilitates wired and/or wireless transmission of video or still image data from the CIT image capture device 606 to the handheld cable housing 608.

In some implementations, the coupling 614 facilitates transmission of light of an illumination source from the handheld cable housing 608 to the rotatable endoscope assembly 601. For example, the rotatable endoscope assembly 601 includes an illumination element (not shown), such as the illumination element 515 (e.g., a light pipe or fiber optic bundle) to direct light from the handheld cable housing 608 through the coupling 614 to the CIT image capture device 606. For example, the CIT image capture device 606 comprises illumination optics, such as the illumination optics 516 described above, to direct the light from the handheld cable housing 608 to illuminate a scene being captured by the CIT image capture device 606.

In some implementations the coupling 614 is a releasable coupling such that the endoscope assembly 601 is releasably removed from the handheld cable housing 608. Therefore, each of the rotatable endoscope assembly 601 and the handheld cable housing 608 may be separately cleaned. Additionally, different rotatable endoscope assemblies 601 may be attached to the handheld cable housing 608, such as rotatable endoscope assemblies 601 with different angles for the angle 514 or with different tools, features, or functions. For example, the rotatable endoscope assembly 601 may be a 30 degree laparoscope, a zero degree laparoscope, a 30 degree cystoscope, or any other such tool.

In some implementations the coupling 614 is a fixed coupling such that the endoscope assembly 601 is not releasable from the handheld cable housing 608.

The handheld cable housing 608 comprises a grip 616. In some implementations, the grip 616 may include contours or other ergonomic features to facilitate ease of use and handling of the endoscopic image capture device 600. In implementations where the rotatable endoscope shaft 602 is releasably attached to the handheld cable housing 608, the grip 616 may additionally include a release button (not shown) for releasing a locking mechanism (not shown) that holds the rotatable endoscope shaft 602 to the handheld cable housing 608.

The handheld cable housing 608 comprises a control surface 618 with one or more control buttons (not shown) positioned thereon. The control buttons are one or more of a physical button, a soft button on a touch screen, a switch, a touch pad, a scroll wheel, a directional pad, or any other user input device. The control surface 618 of the handheld cable housing 608 is positioned such that the one or more control buttons are easily accessible while a user is holding the grip 616 even when the rotatable endoscope assembly 601 is rotated relative to the handheld cable housing 608. That is, the control surface 618 with the one or more control buttons remain fixed even when the rotatable endoscope assembly 601 is rotated relative to the handheld cable housing 608.

In the example shown in FIGS. 6A and 6B, the control surface 618 is positioned on a top surface of the handheld cable housing 608 that extends away from the grip 616. However, the control surface 618 may be positioned anywhere on the handheld cable housing 608, such as on a side surface, or a surface that extends out from the handheld cable housing 608 (e.g., extends out parallel to the grip 616 to facilitate ease of viewing the control surface 618 when a user is positioned behind the grip 616), or any other surface of the handheld cable housing 608.

In some implementations, the control surface 618 is a movable surface to permit a user positioning the control surface 618 in a desired orientation to facilitate ergonomic activation of the one or more control buttons thereon. For example, the control surface 618 may be tilted up or otherwise oriented in different directions to facilitate ergonomic activation of one or more control buttons.

The handheld cable housing 608 comprises a connector cable 620 that facilitates wired and/or wireless transmission of data and power between the handheld cable housing 608 and an external device, such as the electronics cart 56 or the electronics cart 24 discussed above. The connector cable 620 comprises a socket (not shown) to facilitate coupling the handheld cable housing 608 with the external device. For example, the connector cable 620 supplies power received from the external device to the handheld cable housing 608. The connector cable 620 supplies data from the handheld cable housing 608 to the external device. For example, the handheld cable housing 608 supplies video or still image data from the CIT image capture device 606 to the external device. Similarly, the handheld cable housing 608 supplies one or more control signals upon selection of a control button on the control surface 618. For example, the control signal provides instructions to turn on or off an illumination source, capture a still image from the image sensor, start/stop video recording, define an image horizon, turn on or off horizonal image alignment, or perform any other control function for operation of the endoscopic image capture device 600.

In some implementations, the connector cable 620 facilitates transmission of light from an illumination source in the external device to the handheld cable housing 608. For example, handheld cable housing 608 includes an illumination element (not shown), such as a light pipe, single optical fiber, or fiber optic bundle, to receive light from the connector cable 620 and direct the received light through the coupling 614 to the CIT image capture device 606, as described above. Likewise, the handheld cable housing 608 includes an illumination element (not shown), such as a light pipe, single optical fiber, or fiber optic bundle, to receive light from the external device and direct the received light through the socket to the handheld cable housing 608. In some implementations, the illumination source is positioned in the handheld cable housing 608.

In some implementations, the illumination element of the handheld cable housing 608 is an extension of the illumination element of the connector cable 620. For example, upon assembly of the handheld cable housing 608 to the connector cable 620, a portion of the illumination element of the connector cable 620 extends into the handheld cable housing 608 to the coupling 614.

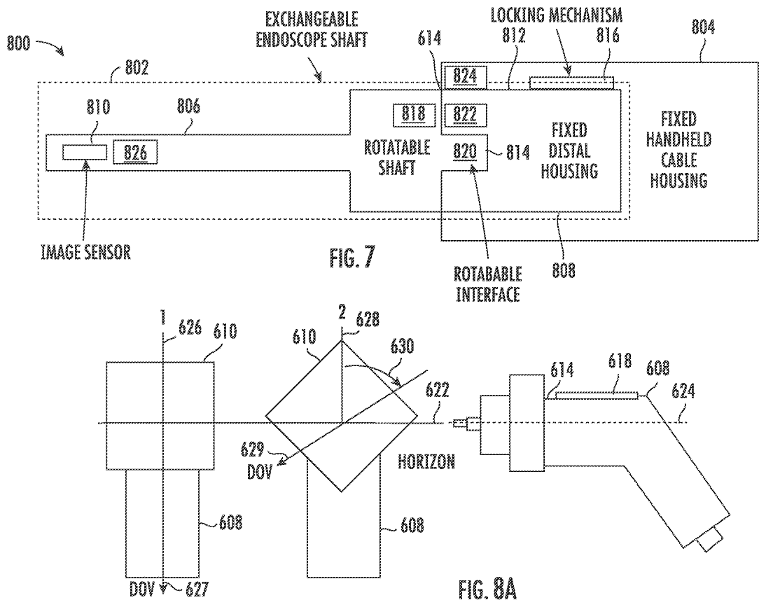

FIG. 7 is a block diagram of elements of an endoscopic image capture device 800. In some implementations, the endoscopic image capture device 800 is implemented as the endoscopic image capture device 600 described above, where like numerals represent like parts.

The endoscopic image capture device 800 has a rotatable endoscope assembly 802 (e.g., rotatable endoscope shaft), such as the rotatable endoscope assembly 601 described above, that is releasably or fixedly coupled to a fixed handheld cable housing 804, such as the handheld cable housing 608.

The rotatable endoscope assembly 802 comprises a rotatable endoscope shaft 806 and a fixed distal housing 808. An image sensor 810, such as the CIT image capture device 606 or the CIT image capture device 500 described above, is positioned on a distal end of the rotatable endoscope shaft 806. The fixed distal housing 808 is located at a proximal end of the rotatable endoscope assembly 802. The fixed distal housing 808 is sized and configured to be releasably received in a socket 812 of the fixed handheld cable housing 804. The rotatable endoscope shaft 806 is rotatable with respect to the fixed distal housing 808 about a rotatable interface 814. The rotatable endoscope assembly 802 and the fixed handheld cable housing 804 are releasably affixed to each other by a locking mechanism 816. The rotatable endoscope assembly 802 is released from the fixed handheld cable housing 804 upon unlocking the locking mechanism 816, such as upon pressing a button (not shown) that unlocks the locking mechanism 816.

While the example shown in FIG. 7 provides the rotatable endoscope assembly 802 is releasably coupled to a fixed handheld cable housing 804, in some implementations, the rotatable endoscope assembly 802 may be fixedly coupled to the handheld cable housing 804. As such, the fixed distal housing 808 and the locking mechanism 816 may be omitted and the rotatable interface 814 of the rotatable endoscope assembly 802 is directly coupled to the handheld cable housing 804 via the socket 812.

The endoscopic image capture device 800 comprises one or more angular position sensors that measures an angular offset relative to a defined image horizon as the rotatable endoscope assembly 802 is rotated relative to the handheld cable housing 804.

In some implementations, the defined image horizon is defined with respect to the rotatable endoscope assembly 802 or the handheld cable housing 804. As such, a first angular position sensor is positioned at a first angular position sensor location 818 at the coupling 614 and is configured to measure an angular offset of the rotatable endoscope assembly 802 relative to the handheld cable housing 804. As shown, the first angular position sensor location 811 is located at a distal end of the rotatable endoscope shaft 806, such as at the coupling 614. The angular position sensor may be a Hall effect sensor, an encoder (e.g., mechanical, optical, magnetic, electromagnetic induction), rotary potentiometer, resolver, and/or any other angular measurement sensor.

Alternatively or additionally, the angular position sensor may be located at a second angular position sensor location 820 within or on the rotatable interface 814, at a third angular position sensor location 822 within or on the fixed distal housing 808 (e.g., a proximal end of the rotatable endoscope assembly 802 that extends within the handheld cable housing 804 and is held in a fixed orientation relative to the handheld cable housing 804), at a fourth angular position sensor location 824 within or on the handheld cable housing 804, and/or any other location suitable for measurement of an angular offset between the rotatable endoscope assembly 802 and the handheld cable housing 804..

While the first angular position sensor location 818 is depicted as located at the coupling 614, the first angular position sensor location 818 may be located anywhere within or on the rotatable endoscope shaft 806. While the third angular position sensor location 822 is depicted as located at the coupling 614, the third angular position sensor location 822 may be located anywhere within or on the fixed distal housing 808. While the fourth angular position sensor location 824 is depicted as located at the coupling 614, the fourth angular position sensor location 824 may be located anywhere within or on the handheld cable housing 804.

In some implementations, the angular position sensor is located at a plurality of the angular position sensor locations 818-824. For example, with a Hall effect sensor, a ring magnet may be positioned at a first of the plurality of the angular position sensor locations 818-824 and a Hall effect sensor may be positioned at a second of the plurality of the angular position sensor locations 818-824. For example, the ring magnet may be positioned at either of the first or second angular position sensor locations 818-820 and the Hall effect sensor may be positioned at the third or fourth angular position sensor locations 822-824, or vice versa.

In some implementations, rather than the defined image horizon being defined with respect to the rotatable endoscope assembly 802 or the handheld cable housing 804, the defined image horizon is based on a sensed direction of gravity. In such implementations, the one or more angular position sensors include a gravity sensor to measure the angular offset relative to gravity. For example, the defined image horizon may be the sensed direction of gravity, or some angle based on the sensed direction of gravity, such as a direction orthogonal to the sensed direction of gravity.

In some implementations, the gravity sensor is a gyroscope, magnetometer, and/or linear acceleration sensor, or any other gravity sensor. For example, an accelerometer provides a multi-axis measurement of proper acceleration. Based on measurements form a calibration step (e.g., proper acceleration with the endoscopic image capture device 800 in a calibration orientation) or in combining the measurement of proper acceleration with one or more other sensors (e.g., magnetometer), a magnitude of the proper acceleration due to gravity on one or more of the axes can be determined. Based on the relative magnitude of gravity on each of the axis, an orientation of the endoscopic image capture device 800 relative to gravity can be calculated. While an example of gravity measurement is provided above, any other method or sensor for measuring gravity is contemplated by this disclosure.

In some implementations, the angular offset is the resultant orientation of the endoscopic image capture device 800 with respect to gravity, the relative magnitude of gravity on each axis of orientation, an offset from the sensed direction of gravity (e.g., orthogonal from the sensed direction of gravity), or any other measurement indicative of the orientation of the endoscopic image capture device 800 relative to gravity. In some implementations, the direct measurements of the gravity sensor (e.g., one or more accelerometer, magnetometer, and/or gyroscope measurements) are supplied as the angular offset and used to calculate the orientation of the endoscopic image capture device 800 on an external device, such as the electronics cart 56 or electronics cart 24.

In an example, a gravity sensor is positioned at a fifth angular position sensor location 826 located at a distal end or anywhere along the rotatable endoscope shaft 806 of the rotatable endoscope assembly 802, such as at the CIT image capture device 810.

Therefore, the gravity sensor measures an orientation of the CIT image capture device 810 relative to gravity as the angular offset. That is, the gravity sensor directly measures an orientation of the CIT image capture device 810 relative to gravity so that captured images and video can be rotated to maintain a gravity-based horizon.

In another example, one gravity sensor is positioned at the first, second, or fifth angular position sensor locations 818-120, 826 and a second gravity sensor is positioned at the third or fourth angular position sensor locations 822-824 in the handheld cable housing 804 or the fixed distal housing 808. Therefore, an orientation of the CIT image capture device 810 or any other portion of the rotatable endoscope shaft 806 and an orientation of the handheld cable housing 804 or the fixed distal housing 808 relative to gravity can be determined. The angular offset is a difference between the orientations relative to gravity of the CIT image capture device 810 or any other portion of the rotatable endoscope shaft 806 and an orientation of the handheld cable housing 804 or the fixed distal housing 808 relative to gravity.

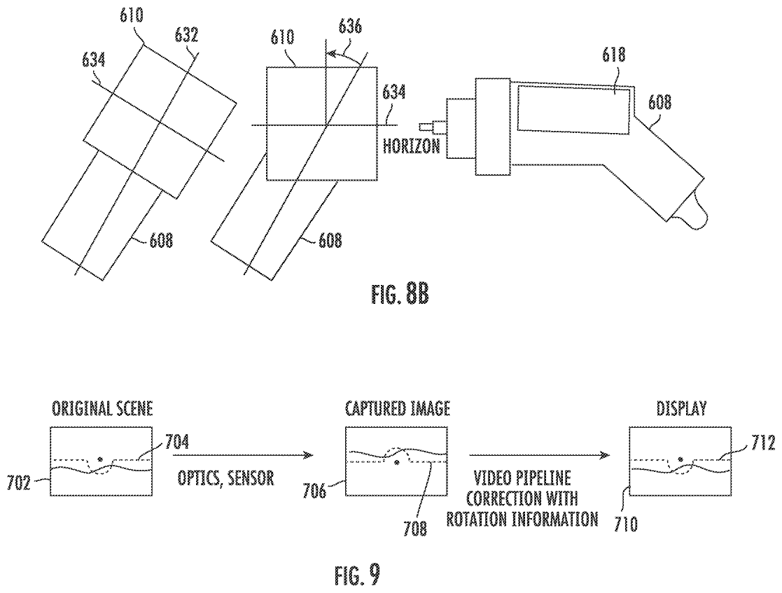

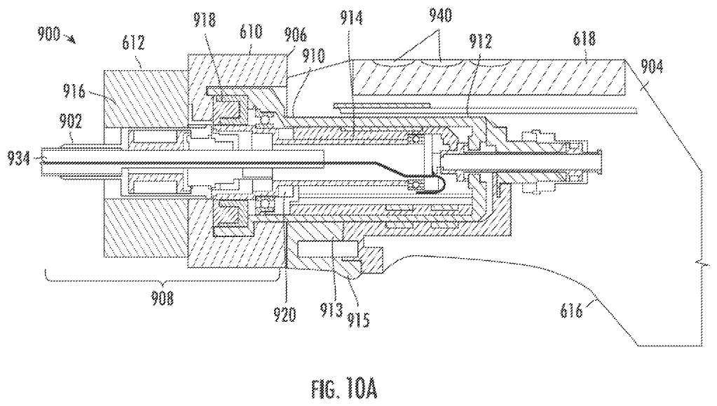

In some implementations, the defined image horizon may be defined based on an orientation of the handheld cable housing 804 or the rotatable endoscope assembly 802, but the angular offset between the two is determined based on a relative orientation of each with respect to gravity, such as described below in the examples of FIGS. 8A and 8B.

In some implementations, rather than positioning one or more gravity sensors in the handheld cable housing 804, the one or more gravity sensors are positioned in a proximal end of the rotatable endoscope assembly 802 that extends within the handheld cable housing 804 and is held in a fixed orientation relative to the handheld cable housing 804, such as at the third angular sensor location 822 in the fixed distal housing 808. For example, with a replaceable rotatable endoscope assembly 802, the one or more gravity sensors are positioned in or on the fixed distal housing 808 (e.g., a fixed proximal end of the rotatable endoscope assembly 802 that connects with the handheld cable housing 804).

In a further example, one or more gravity sensors are positioned in or on the handheld cable housing 804 to measure an orientation of the handheld cable housing 804 relative to gravity. Additionally, an angular position sensor positioned at the coupling 614 measures an angular offset of the rotatable endoscope assembly 802 relative to the handheld cable housing 608. Therefore, an orientation of the rotatable endoscope assembly 802, hence an orientation of the CIT image capture device 810, relative to gravity can be determined based on a combination of an angular offset measurement from one or more gravity sensors and an angular offset measurement from an angular position sensor.

Similarly, one or more gravity sensors are positioned in the rotatable endoscope shaft 806 to measure its orientation relative to gravity. Additionally, an angular position sensor positioned at the coupling 614 measures an angular offset of the rotatable endoscope shaft 806 relative to the handheld cable housing 804. Therefore, an orientation of the handheld cable housing 804 relative to gravity can be determined based on a combination of an angular offset measurement from one or more gravity sensors and an angular offset measurement from an angular position sensor.

In some implementations, the gravity sensors described above may include two or more gravity sensors mounted at a specific orientation to one another, for example two gravity sensors mounted orthogonal to each other, to prevent gimbal lock. In some implementations more than two gravity sensors may be used. In some implementations, the two or more gravity sensors are mounted at orientations other than at 90°. For example, a gravity sensor in the handheld cable housing 608 includes two gravity sensors mounted orthogonal to each other in the handheld cable housing 608.

As shown in FIG. 8A, a horizon 622 is defined with respect to the handheld cable housing 608. Specifically, the horizon 622 is a horizontal midline plane 624 of the handheld cable housing 608 at the coupling 614. Stated another way, the horizon 622 is orthogonal to a vertical midline plane of the handheld cable housing 608 and parallel to a longitudinal axis of the handheld cable housing 608.

In this example, the rotatable endoscope assembly 601 includes the CIT image capture device 606 with the angle 514 greater than 0° (e.g., a 30° tip). In a first configuration, the rotatable endoscope assembly 601 is positioned at the reference position with respect to the handheld cable housing 608, and hence with respect to the horizon 622. Therefore, the CIT image capture device 606 is able to capture images from a first DOV 627.

In a second configuration, the rotatable endoscope assembly 601 is rotated at an angle 630 from the reference position while the handheld cable housing 608 remains fixed. Therefore, the CIT image capture device 606 is able to capture images from a second DOV 629. Accordingly, the first angular position sensor measures the angle 630 as the angular offset. As described in more detail with reference to FIG. 9, with horizontal image alignment turned on, image data captured by the CIT image capture device 606 is rotated based on the measured angle 630 so that a displayed image aligns to the image horizon defined by the handheld cable housing 608.

In another example shown in FIG. 8B, the rotatable endoscope assembly 601 includes the CIT image capture device 606 with the angle 514 of 0° (e.g., a zero-degree tip or zero-degree optics). Therefore, as the rotatable endoscope assembly 601 is rotated with respect to the handheld cable housing 608, a DOV of the CIT image capture device 606 remains the same, but is rotated.

In some implementations, it may not be desirable enable horizontal image alignment. For example, with zero-degree optics, the horizontal image alignment can be turned off either manually upon selection of a control button on the control surface 618 or automatically upon detection of the rotatable endoscope assembly 601 with zero-degree optics. In either case, a user manually manipulates the lever 610 (e.g., alignment wheel) to maintain an image horizon. For example, the user rotates the lever 610 so that the rotatable endoscope assembly 601 is aligned with the horizon.

In this case, rather than defining the horizon with respect to the handheld cable housing 608, the horizon is defined with respect to the rotatable endoscope assembly 601.

For example, as shown in FIG. 8B, a horizon 634 is defined with respect to the rotatable endoscope assembly 601. Specifically, the horizon 634 is a horizontal midline plane of the rotatable endoscope assembly 601 at the coupling 614. Stated another way, the horizon 634 is orthogonal to a vertical midline plane of the rotatable endoscope assembly 601 and parallel to a longitudinal axis of the rotatable endoscope assembly 601.

In some implementations, the vertical midline plane of the rotatable endoscope assembly 601 intersects the reference position. Therefore, the horizon 634 is orthogonal to a midline plane that intersects the reference position of the rotatable endoscope assembly 601 and parallel to a longitudinal axis of the rotatable endoscope assembly 601. M ore generally, the horizon 634 is orthogonal to a midline plane that intersects a midpoint of a path of motion of the rotatable endoscope assembly 601 and parallel to a longitudinal axis of the rotatable endoscope assembly 601.

In a first configuration, the rotatable endoscope assembly 601 is positioned at the reference position with respect to the handheld cable housing 608, where both the rotatable endoscope assembly 601 and the handheld cable housing 608 are oriented at an angle (e.g., inclined sideways).

In a second configuration, the rotatable endoscope assembly 601 is manually rotated at an angle 636 from the reference position while the handheld cable housing 608 remains fixed. Accordingly, a user manually maintains the horizontal image alignment. Because the handheld cable housing 608 is oriented at an angle, the control surface 618 may be more visible and/or accessible for selection of one or more control buttons for certain procedures than when the handheld cable housing 608 is oriented vertically.

In the examples shown in FIGS. 8A & 8B, the reference position is a midpoint position in a path of motion of the rotatable endoscope assembly 601. The rotatable endoscope assembly 601 may be rotatable at angles of +/−180° relative to the midpoint position or at any subset of angles thereof. At the reference position, the angular offset is measured as 0°.

Other reference positions are contemplated by this disclosure, such as a start position in a path of motion where the rotatable endoscope assembly 601 is rotatable +360° relative to the start position or at any subset of angles thereof. In another example, the reference position is an end position in a path of motion where the rotatable endoscope assembly 601 is rotatable −360° relative to the end position or at any subset of angles thereof. More generally, the reference position is at any location along a path of motion where the rotatable endoscope assembly 601 is rotatable across any subset of angles +/−360° from the reference position. In some implementations, the rotatable endoscope assembly 601 does not include a stop such that the rotatable endoscope assembly can rotate in either direction without limit (e.g., continuously rotatable).

In the examples provided above, the horizon is defined based on the handheld cable housing 608, based on the rotatable endoscope assembly 601, relative to gravity as sensed at a distal end of the rotatable endoscope assembly 601 (e.g., at the CIT image capture device 606), relative to gravity as sensed by a fixed proximal end of the rotatable endoscope assembly 601 that connects with the handheld cable housing 608, or relative to gravity as sensed by the handheld cable housing 608. The angular offset is measured with an angular position sensor, such as at the coupling 614, and/or one or more gravity sensors positioned in the rotatable endoscope assembly 601 and/or the handheld cable housing 608, such as described with respect to FIG. 7.

In another example, the horizon is user-defined. For example, upon orienting the endoscopic image capture device 600 in a desired orientation, a control button on the control surface 618 is activated (e.g., pressed) to define the horizon as the desired orientation. Thereafter, any movement from the desired orientation is measured as the angular offset. For example, movement of the rotatable endoscope assembly 601 and/or the handheld cable housing 608 from the desired orientation is measured using the angular position sensor, such as at the coupling 614, and/or one or more gravity sensors positioned in the rotatable endoscope assembly 601 and/or the handheld cable housing 608, as described above.

In various implementations, a button on the control surface 618 is selectable to change an operating mode of the endoscopic image capture device 600 for how the horizon is defined and the angular offset is measured. For example, each press of the button may toggle through different modes of how the horizon is defined and the angular offset is measured.

Each of the modes corresponds to one of the examples for how the horizon is defined and the angular offset is measured, as described above. In some implementations, a subset of the examples may be used for the set of operating modes of the endoscopic image capture device 600. In some implementations, the endoscopic image capture device 600 includes an operating mode where no horizon is defined and no angular offset is measured.

Each of the features of the endoscopic image capture device 600 described above may be used separately or in combination with one another or other features described throughout this disclosure. Various modifications and additions to the endoscopic image capture device 600 are readily discernable by those of ordinary skill in the art and are contemplated by this disclosure. For example, alternative illumination, filtering, optical assembly, focus manipulation, and image sensor features known to those of ordinary skill in the art are contemplated by this disclosure.

FIG. 9 is a sequence diagram of image processing operations to maintain horizontal image alignment. A first view 702 shows an original scene oriented with respect to a defined horizon 704. The defined horizon 704 may be defined based on any one or a combination of the examples described above for defining an image horizon.

A picture 706 of the first view 702 is captured. In some implementations, the picture 706 is captured with the optical assembly 506 and the image sensor 510 of the chip-in-tip (CIT) image capture device 500 described. In some implementations, the picture 706 is captured by the endoscopic image capture device 600 described above with the CIT image capture device 606. Regardless, the picture 706 of the first view 702 is captured using equipment oriented at an angular offset relative to the defined horizon 704. In the example shown, an image horizon 708 of the picture 706 is at an angular offset of 180° with respect to the defined horizon 704. For example, the picture 706 may be captured by the image capture device 600 oriented as shown in FIG. 6B.

A measurement of the angular offset and the picture 706 communicated to an image processor to rotate the picture 706 based on the angular offset to maintain a constant image horizon in a displayed image 710. Therefore, the displayed image 710 has displayed image horizon 712 that matches the defined horizon 704.

For example, the endoscopic image capture device 600 communicates the picture 706 as a still image or video stream to the electronics cart 56, electronics cart 24, or processor 58 to rotate the picture 706 based on the angular offset. Specifically, images captured by the CIT image capture device 606 and corresponding measurements of the angular offset as measured by one or more angular position sensors are communicated from the endoscopic image capture device 600 via the connector cable 620 to electronics cart 56 or electronics cart 24 for processing (e.g., image rotation). Once processed, the rotated images are displayed on a display with a constant image horizon, such as on the display 60, the display 25, or the display area 31.

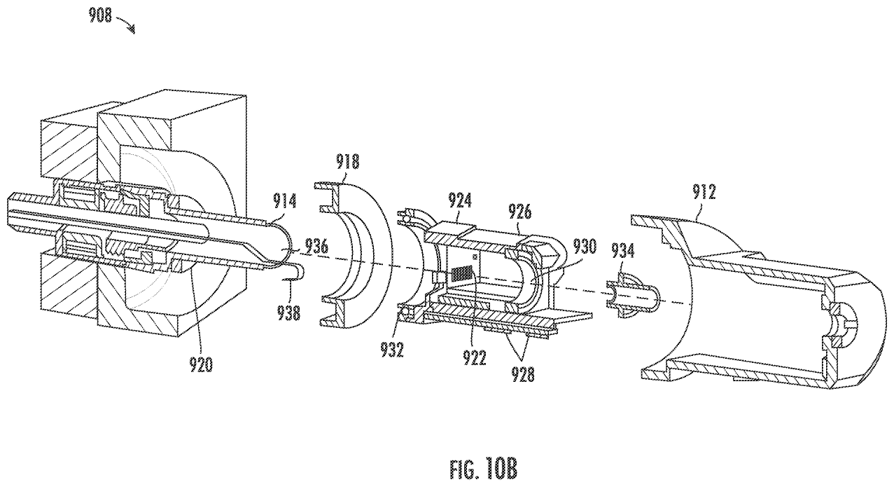

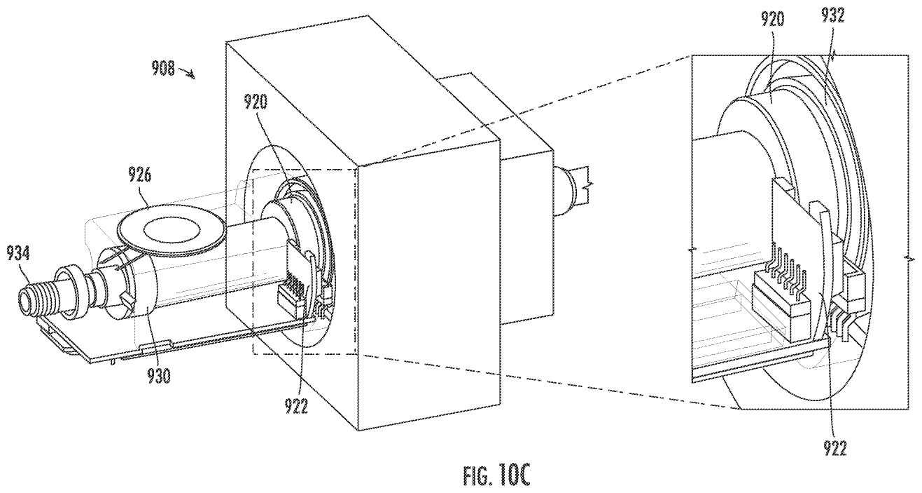

FIG. 10A is a cross-sectional view of an example endoscopic image capture device 900 showing a coupling between a rotatable endoscope assembly 902 and a fixed handheld cable housing 904. FIG. 10B is an exploded view of the rotatable endoscope assembly 902 of FIG. 10A. FIG. 10C is a perspective view of the rotatable endoscope assembly 902 of FIG. 10A showing details of an angular position sensor for measuring rotation between the rotatable endoscope assembly 902 and the fixed handheld cable housing 904.

In some implementations, the endoscopic image capture device 900 is implemented as the endoscopic image capture device 600 or endoscopic image capture device 800 described above, where like numbers refer to like parts. Accordingly, the rotatable endoscope assembly 902 is implemented as the rotatable endoscope assembly 601 or the rotatable endoscope assembly 802. Likewise, the handheld cable housing 904 is implemented as the handheld cable housing 608 or the handheld cable housing 804.

As shown in FIG. 10A a bold dashed line shows an interface 906 between the rotatable endoscope assembly 902 (e.g., rotatable endoscope shaft) and the fixed handheld cable housing 904. The rotatable endoscope assembly 902 comprises a rotatable endoscope shaft 908, such as the rotatable endoscope shaft 806, and a fixed distal housing 910, such as the fixed distal housing 808. The fixed distal housing 910 is located at a proximal end of the rotatable endoscope assembly 902.

The fixed distal housing 910 is sized and configured to be releasably received in a socket 912 of the fixed handheld cable housing 904. A lock 913 maintains the rotatable endoscope shaft 908 within the socket 912 of the fixed handheld cable housing 904. In various implementations, the lock 913 is biased in a locked configuration. A release lever 915 is selectable for configuring the lock 913 in an unlocked configuration to facilitate removing the rotatable endoscope shaft 908 from the socket 912 of the fixed handheld cable housing 904.

The rotatable endoscope shaft 908 is rotatable with respect to the fixed distal housing 910 about a rotatable interface 914. For example, the lever 610 is coupled to the rotatable interface 914 for rotation of the rotatable endoscope shaft 908 relative to the fixed distal housing 910.

In some implementations, a radial shaft seal ring 918 seals an interior volume of the fixed distal housing 910 to prevent ingress of an environment surrounding the endoscopic image capture device 900.

The rotatable endoscope assembly 902 has an angular position sensor located in the fixed distal housing 910. In the example shown, the angular position sensor includes a ring magnet 920 coupled to the rotatable interface 914 and configured to rotate with the rotatable endoscope shaft 908. A Hall effect sensor 922 is positioned in the fixed distal housing 910 to detect a changing magnetic field of the ring magnet 920 upon rotation with the rotatable endoscope shaft 908.

The rotatable endoscope assembly 902 has an electronics assembly 924 coupled to the fixed distal housing 910 and configured to communicate data and power with the handheld cable housing 904, best shown in FIG. 10B. The electronics assembly 924 has a power interface 926, such as a receiver induction coil. The electronics assembly 924 also has one or more data interfaces 928, such as transmitter induction coils. The electronics assembly 924 also has a ferrule 934 configured to receive light from an illumination source via the fixed handheld cable housing 904.

While transmitter and receiver induction coils are in the example shown for wireless power and data transmission, any wired and/or wireless power and/or data interface may be used. Because the example shown uses wireless data and power transmission, the rotatable endoscope assembly 902 is a sealed system such that the rotatable endoscope is able to be cleaned and sterilized, such as in an autoclave.

In some implementations, the electronics assembly 924 also has ball bearings coupled between the fixed distal housing 910 and the rotatable interface 914 to facilitate of the rotatable endoscope shaft 908 relative to the fixed distal housing 910. A proximal bearing 930 is positioned at a proximal end of the rotatable interface 914. A distal bearing 932 is positioned at a distal end of the rotatable interface 914.

The rotatable endoscope shaft 908 has a lumen 936 configured for communication of power, data, and illumination with an image capture device at a distal end of the rotatable endoscope shaft 908, such as the CIT image capture device 606 or CIT image capture device 500. In the example shown, a flat flex 938 is provided for communication of data and/or power through the lumen 936. In some implementations, an illumination element (not shown), such as a light pipe or fiber optic bundle, communicates light received at the ferrule 934 from an illumination source through the lumen 936.

The handheld cable housing 904 has the control surface 618 with a plurality of control buttons 940 positioned thereon. The control buttons 940 control one or more operating functions (e.g., turn on or off illumination, change illumination source, turn on or off horizontal image alignment, change defined horizon, capture still image, etc.), as described above.

Each of the features of the endoscopic image capture device 900 described above may be used separately or in combination with one another or other features described throughout this disclosure. Various modifications and additions to the endoscopic image capture device 900 are readily discernable by those of ordinary skill in the art and are contemplated by this disclosure. For example, alternative illumination, filtering, optical assembly, focus manipulation, and image sensor features known to those of ordinary skill in the art are contemplated by this disclosure.

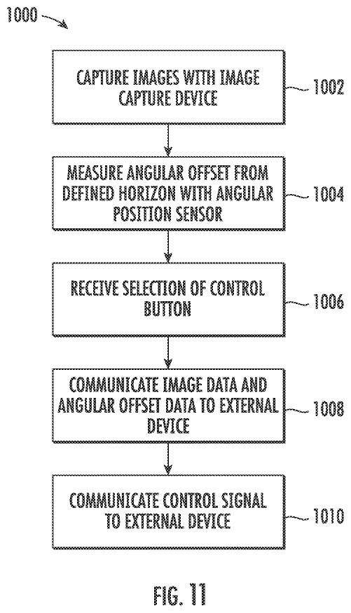

FIG. 11 is a flowchart 1000 of operation of an image capture device according to various implementations described herein. In some implementations, the image capture device is the endoscopic image capture device 900, the endoscopic image capture device 800, or the endoscopic image capture device 600 described above. As with the systems described above, the image capture device has an image capture assembly with an image sensor positioned therein that is rotatably attached with a control assembly with a control surface with one or more control buttons thereon. For example, the image capture assembly may be the rotatable endoscope assembly 601, the rotatable endoscope assembly 802, or the rotatable endoscope shaft 908, described above. Likewise, the control assembly may be the handheld cable housing 608, the fixed handheld cable housing 804, or the fixed handheld cable housing 904, described above. Therefore, as the image capture assembly is rotated relative to the control assembly, the one or more control buttons remain readily accessible.

At 1002 still or video image are captured by the image capture device. For example, still or video images are captured by the CIT image capture device 500, the CIT image capture device 606, or the CIT image capture device 810.

At 1004, the image capture device measures an angular offset from a defined horizon with one or more angular position sensors. For example, the one or more angular position sensors are one or more of a hall effect sensor, an encoder (e.g., mechanical, optical, magnetic, electromagnetic induction), a rotary potentiometer, a resolver, any other angular measurement sensor, a gyroscope, a magnetometer, a linear acceleration sensor, and/or any other gravity sensor. The angular position sensor may be positioned at one or more of the image capture assembly, the control assembly, and/or a coupling between the image capture assembly and the control assembly.

At 1006, the control assembly receives a selection of a control button on the control surface. For example, the control button generates a control signal for performing one or more operating functions (e.g., turn on or off illumination, change illumination source, turn on or off horizontal image alignment, change defined horizon, capture still image, etc.).

At 1008, the control assembly communicates image data captured by the image capture device and angular offset data measured by the one or more angular position sensors to an external device. For example, a connector cable of the control assembly, such as the connector cable 620 described above, facilitates wired and/or wireless transmission of data and power between the control assembly and the external device, such as the electronics cart 56 or the electronics cart 24, as discussed above.

Likewise, at 1010, the control assembly communicates the control signal to the external device responsive to receiving the selection of the control button at 1006. For example, the connector cable, such as the connector cable 620, facilitates wired and/or wireless transmission of the control signal to the external device, such as the electronics cart 56 or the electronics cart 24.

FIG. 12 is a flowchart of operation of an image processor according to various implementations described herein. In some implementations, the image processor is the electronics cart 56, electronics cart 24, or processor 58.

At 1102, the image processor receives image data and angular offset data from an image capture device, such as the image capture device of FIG. 11 (e.g., the endoscopic image capture device 900, the endoscopic image capture device 800, or the endoscopic image capture device 600 described above).

At 1104, the image processor rotates the image data based on the angular offset data. The rotated image data is displayed at 1106 so that a constant image horizon is maintained on the displayed image data. For example, the rotated images are displayed on a display with a constant image horizon, such as on the display 60, the display 25, or the display area 31.

At 1108, the image processor receives and processes a control signal. For example, the control signal is received from a control assembly, such as the control assembly of FIG. 11. The image processor performs one or more operations based on processing of the control signal. For example, the image processor operates to turn on or off an illumination source supplied by the image processor, capture a still image from the received image data, start/stop video recording from the received image data, define an image horizon (e.g., toggle through different modes of how the horizon is defined and the angular offset is measured, as described above), turn on or off horizonal image alignment, or perform any other control function for operation of a connected image capture device.

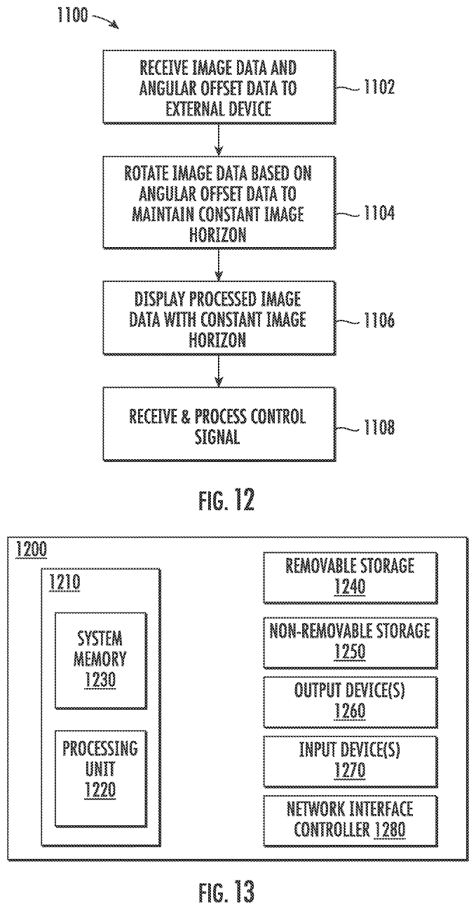

It should be appreciated that the logical operations described herein with respect to the various figures may be implemented (1) as a sequence of computer implemented acts or program modules (i.e., software) running on a computing device (e.g., the computing device described in FIG. 13), (2) as interconnected machine logic circuits or circuit modules (i.e., hardware) within the computing device and/or (3) a combination of software and hardware of the computing device. Thus, the logical operations discussed herein are not limited to any specific combination of hardware and software. The implementation is a matter of choice dependent on the performance and other requirements of the computing device. Accordingly, the logical operations described herein are referred to variously as operations, structural devices, acts, or modules. These operations, structural devices, acts and modules may be implemented in software, in firmware, in special purpose digital logic, and any combination thereof. It should also be appreciated that more or fewer operations may be performed than shown in the figures and described herein. These operations may also be performed in a different order than those described herein.

Referring to FIG. 13, an example computing device 1200 upon which embodiments of the invention may be implemented is illustrated. For example, each of the computer processor located on an electronics cart 56 or electronics cart 24, and computer processor 58 described herein may each be implemented as a computing device, such as computing device 1200. It should be understood that the example computing device 1200 is only one example of a suitable computing environment upon which embodiments of the invention may be implemented. Optionally, the computing device 1200 can be a well-known computing system including, but not limited to, personal computers, servers, handheld or laptop devices, multiprocessor systems, microprocessor-based systems, network personal computers (PCs), minicomputers, mainframe computers, embedded systems, and/or distributed computing environments including a plurality of any of the above systems or devices. Distributed computing environments enable remote computing devices, which are connected to a communication network or other data transmission medium, to perform various tasks. In the distributed computing environment, the program modules, applications, and other data may be stored on local and/or remote computer storage media.

In an embodiment, the computing device 1200 may comprise two or more computers in communication with each other that collaborate to perform a task. For example, but not by way of limitation, an application may be partitioned in such a way as to permit concurrent and/or parallel processing of the instructions of the application. Alternatively, the data processed by the application may be partitioned in such a way as to permit concurrent and/or parallel processing of different portions of a data set by the two or more computers. In an embodiment, virtualization software may be employed by the computing device 1200 to provide the functionality of a number of servers that is not directly bound to the number of computers in the computing device 1200. For example, virtualization software may provide twenty virtual servers on four physical computers. In an embodiment, the functionality disclosed above may be provided by executing the application and/or applications in a cloud computing environment. Cloud computing may comprise providing computing services via a network connection using dynamically scalable computing resources. Cloud computing may be supported, at least in part, by virtualization software. A cloud computing environment may be established by an enterprise and/or may be hired on an as-needed basis from a third-party provider. Some cloud computing environments may comprise cloud computing resources owned and operated by the enterprise as well as cloud computing resources hired and/or leased from a third-party provider.

In its most basic configuration, computing device 1200 typically includes at least one processing unit 1220 and system memory 1230. Depending on the exact configuration and type of computing device, system memory 1230 may be volatile (such as random-access memory (RAM)), non-volatile (such as read-only memory (ROM), flash memory, etc.), or some combination of the two. This most basic configuration is illustrated in FIG. 13 by dashed line 1210. The processing unit 1220 may be a standard programmable processor that performs arithmetic and logic operations necessary for operation of the computing device 1200. While only one processing unit 1220 is shown, multiple processors may be present. Thus, while instructions may be discussed as executed by a processor, the instructions may be executed simultaneously, serially, or otherwise executed by one or multiple processors. The computing device 1200 may also include a bus or other communication mechanism for communicating information among various components of the computing device 1200.

Computing device 1200 may have additional features/functionality. For example, computing device 1200 may include additional storage such as removable storage 1240 and non-removable storage 1250 including, but not limited to, magnetic or optical disks or tapes. Computing device 1200 may also contain network connection(s) 1280 that allow the device to communicate with other devices such as over the communication pathways described herein. The network connection(s) 1280 may take the form of modems, modem banks, Ethernet cards, universal serial bus (USB) interface cards, serial interfaces, token ring cards, fiber distributed data interface (FDDI) cards, wireless local area network (WLAN) cards, radio transceiver cards such as code division multiple access (CDMA), global system for mobile communications (GSM), long-term evolution (LTE), worldwide interoperability for microwave access (WiMAX), and/or other air interface protocol radio transceiver cards, and other well-known network devices. Computing device 1200 may also have input device(s) 1270 such as a keyboards, keypads, switches, dials, mice, track balls, touch screens, voice recognizers, card readers, paper tape readers, or other well-known input devices. Output device(s) 1260 such as a printers, video monitors, liquid crystal displays (LCDs), touch screen displays, displays, speakers, etc. may also be included. The additional devices may be connected to the bus in order to facilitate communication of data among the components of the computing device 1200. All these devices are well known in the art and need not be discussed at length here.

The processing unit 1220 may be configured to execute program code encoded in tangible, computer-readable media. Tangible, computer-readable media refers to any media that is capable of providing data that causes the computing device 1200 (i.e., a machine) to operate in a particular fashion. Various computer-readable media may be utilized to provide instructions to the processing unit 1220 for execution. Example tangible, computer-readable media may include, but is not limited to, volatile media, non-volatile media, removable media and non-removable media implemented in any method or technology for storage of information such as computer readable instructions, data structures, program modules or other data. System memory 1230, removable storage 1240, and non-removable storage 1250 are all examples of tangible, computer storage media. Example tangible, computer-readable recording media include, but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

It is fundamental to the electrical engineering and software engineering arts that functionality that can be implemented by loading executable software into a computer can be converted to a hardware implementation by well-known design rules. Decisions between implementing a concept in software versus hardware typically hinge on considerations of stability of the design and numbers of units to be produced rather than any issues involved in translating from the software domain to the hardware domain. Generally, a design that is still subject to frequent change may be preferred to be implemented in software, because re-spinning a hardware implementation is more expensive than re-spinning a software design. Generally, a design that is stable that will be produced in large volume may be preferred to be implemented in hardware, for example in an application specific integrated circuit (ASIC), because for large production runs the hardware implementation may be less expensive than the software implementation. Often a design may be developed and tested in a software form and later transformed, by well-known design rules, to an equivalent hardware implementation in an application specific integrated circuit that hardwires the instructions of the software. In the same manner as a machine controlled by a new ASIC is a particular machine or apparatus, likewise a computer that has been programmed and/or loaded with executable instructions may be viewed as a particular machine or apparatus.

In an example implementation, the processing unit 1220 may execute program code stored in the system memory 1230. For example, the bus may carry data to the system memory 1230, from which the processing unit 1220 receives and executes instructions. The data received by the system memory 1230 may optionally be stored on the removable storage 1240 or the non-removable storage 1250 before or after execution by the processing unit 1220.

It should be understood that the various techniques described herein may be implemented in connection with hardware or software or, where appropriate, with a combination thereof. Thus, the methods and apparatuses of the presently disclosed subject matter, or certain aspects or portions thereof, may take the form of program code (i.e., instructions) embodied in tangible media, such as floppy diskettes, CD-ROMs, hard drives, or any other machine-readable storage medium wherein, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs may implement or utilize the processes described in connection with the presently disclosed subject matter, e.g., through the use of an application programming interface (API), reusable controls, or the like. Such programs may be implemented in a high-level procedural or object-oriented programming language to communicate with a computer system. However, the program(s) can be implemented in assembly or machine language, if desired. In any case, the language may be a compiled or interpreted language and it may be combined with hardware implementations.

Embodiments of the methods and systems may be described herein with reference to block diagrams and flowchart illustrations of methods, systems, apparatuses and computer program products. It will be understood that each block of the block diagrams and flowchart illustrations, and combinations of blocks in the block diagrams and flowchart illustrations, respectively, can be implemented by computer program instructions. These computer program instructions may be loaded onto a general-purpose computer, special purpose computer, or other programmable data processing apparatus to produce a machine, such that the instructions which execute on the computer or other programmable data processing apparatus create a means for implementing the functions specified in the flowchart block or blocks.

These computer program instructions may also be stored in a computer-readable memory that can direct a computer or other programmable data processing apparatus to function in a particular manner, such that the instructions stored in the computer-readable memory produce an article of manufacture including computer-readable instructions for implementing the function specified in the flowchart block or blocks. The computer program instructions may also be loaded onto a computer or other programmable data processing apparatus to cause a series of operational steps to be performed on the computer or other programmable apparatus to produce a computer-implemented process such that the instructions that execute on the computer or other programmable apparatus provide steps for implementing the functions specified in the flowchart block or blocks.

Accordingly, blocks of the block diagrams and flowchart illustrations support combinations of means for performing the specified functions, combinations of steps for performing the specified functions and program instruction means for performing the specified functions. It will also be understood that each block of the block diagrams and flowchart illustrations, and combinations of blocks in the block diagrams and flowchart illustrations, can be implemented by special purpose hardware-based computer systems that perform the specified functions or steps, or combinations of special purpose hardware and computer instructions.

While several embodiments have been provided in the present disclosure, it should be understood that the disclosed systems and methods may be embodied in many other specific forms without departing from the spirit or scope of the present disclosure. The present examples are to be considered as illustrative and not restrictive, and the intention is not to be limited to the details given herein. For example, the various elements or components may be combined or integrated in another system or certain features may be omitted or not implemented.

Also, techniques, systems, subsystems, and methods described and illustrated in the various embodiments as discrete or separate may be combined or integrated with other systems, modules, techniques, or methods without departing from the scope of the present disclosure. Other items shown or discussed as directly coupled or communicating with each other may be indirectly coupled or communicating through some interface, device, or intermediate component, whether electrically, mechanically, or otherwise. Other examples of changes, substitutions, and alterations are ascertainable by one skilled in the art and could be made without departing from the spirit and scope disclosed herein.

1-79. (canceled)

80. A surgical system, comprising:
a handheld cable housing with a user input device positioned thereon;
an endoscope shaft with a distal end and a proximal end, wherein the proximal end of the endoscope shaft is releasably coupled to the handheld cable housing at an interface axially aligned with a longitudinal axis of the endoscope shaft, wherein the distal end of the endoscope shaft comprises an image sensor, wherein the endoscope shaft is rotatable relative to the handheld cable housing; and
one or more angular position sensors configured to measure an angular offset relative to a defined image horizon, wherein at least one of the one or more angular position sensors is positioned on the endoscope shaft.

81. The surgical system of claim 80, wherein at least one of the one or more angular position sensors is positioned at a coupling between the endoscope shaft and the handheld cable housing.

82. The surgical system of claim 80, wherein the defined image horizon is relative to the handheld cable housing, and wherein the angular offset is a measurement of an angular rotation of the endoscope shaft relative to the handheld cable housing.

83. The surgical system of claim 82, wherein the defined image horizon is a horizontal midline plane of the handheld cable housing at the coupling or wherein the defined image horizon is orthogonal to a vertical midline plane of the handheld cable housing and parallel to a longitudinal axis of the handheld cable housing.

84. The surgical system of claim 80, wherein the angular offset is a measurement of angular rotation between the defined image horizon and a direction of view of the endoscope shaft.

85. The surgical system of claim 80, wherein the defined image horizon is relative to the endoscope shaft, and wherein the angular offset is a measurement of an angular rotation of the handheld cable housing relative to the endoscope shaft.

86. The surgical system of claim 85, wherein the defined image horizon is a horizontal midline plane of the endoscope shaft at the coupling or wherein the defined image horizon is orthogonal to a vertical midline plane of the endoscope shaft and parallel to a longitudinal axis of the endoscope shaft.

87. The surgical system of claim 80, wherein the defined image horizon is based on a sensed direction of gravity.

88. The surgical system of claim 80, wherein at least one of the one or more angular position sensors is positioned at the distal end of the endoscope shaft or wherein at least one the one or more angular position sensors is positioned at the handheld cable housing.

89. The surgical system of claim 80, wherein the user input device is positioned on a control surface of the handheld cable housing, and wherein the control surface is a top surface of the handheld cable housing.

90. The surgical system of claim 80, wherein the handheld cable housing comprises a socket sized and configured to receive the proximal end of the endoscope shaft, wherein the handheld cable housing comprises a lock configured to maintain the proximal end of the endoscope shaft within the socket of the handheld cable housing, and wherein the lock is biased in a locked configuration.

91. The surgical system of claim 90, wherein the handheld cable housing comprises a release lever selectable to configure the lock in an unlocked configuration for releasing the proximal end of the endoscope shaft within the socket of the handheld cable housing.

92. The surgical system of claim 80, wherein the handheld cable housing comprises a data interface configured to receive image data from the image sensor and the angular offset from the one or more angular position sensors, and wherein the proximal end of the endoscope shaft comprises a corresponding data interface configured to supply image data from the image sensor and the angular offset from the one or more angular position sensors to the handheld cable housing.

93. The surgical system of claim 80, wherein the distal end of the endoscope shaft comprises an optical assembly positioned to receive light incident on a distal face of the endoscope shaft, wherein the distal face is oriented at an angle to the endoscope shaft, and wherein the angle is any angle from 0°-90°.

94. A method comprising:
defining an image horizon relative to an orientation of a surgical system, wherein the surgical system comprises a handheld cable housing with a user input device positioned thereon and a shaft with an image sensor positioned in a distal end of the shaft, wherein the shaft is releasably coupled to the handheld cable housing at an interface axially aligned with a longitudinal axis of the shaft such that the shaft is rotatable relative to the handheld cable housing;
determining an angular offset between the image horizon and a direction of view of the shaft, wherein the angular offset is measured by one or more angular position sensors, and wherein at least one of the one or more angular position sensors is positioned on the shaft; and
transmitting image data captured by the image sensor and angular offset data indicative of the angular offset, the angular offset data for rotation of the image data.

95. A method comprising:
receiving image data captured by an image sensor positioned in a distal end of a shaft, wherein the shaft is releasably coupled to a handheld cable housing at an interface axially aligned with a longitudinal axis of the shaft, wherein a user input device is positioned on the handheld cable housing, wherein the shaft is rotatable relative to the handheld cable housing;
receiving angular offset data indicative of an angular offset relative to a defined image horizon, wherein the angular offset is measured by one or more angular position sensors, and wherein at least one of the one or more angular position sensors is positioned on the shaft;
generating rotated image data based on the angular offset data; and
causing display of the rotated image data.

96. The method of claim 95, further comprising:
receiving a control signal in response to selection of the user input device, wherein the control signal sets the defined image horizon to one of a plurality of image horizons.

97. The method of claim 96, further comprising:
supplying light from an illumination source to the handheld cable housing; and
receiving a second control signal in response to selection of the user input device, wherein the second control signal causes the light from the illumination source to change.

98. The method of claim 97, wherein the second control signal causes the light from the illumination source to turn off or change frequency.

99. The method of claim 95, wherein causing display of the rotated image data comprises transmitting the rotated image data to an external display.
